(19) United States
(12) Patent Application Publication
(10) Pub. No.: US 2011/0092500 A1
(43) Pub. Date: Apr. 21, 2011

(54) SGC STIMULATORS, SGC ACTIVATORS AND COMBINATIONS THEREOF FOR THE TREATMENT OF HEARING IMPAIRMENT

(75) Inventors: Peter Sandner, Wuppertal (DE); Johannes-Peter Stasch, Solingen (DE)

(73) Assignee: BAYER SCHERING PHARMA AKTIENGESELLSCHAFT, Berlin (DE)

(21) Appl. No.: 12/992,083

(22) PCT Filed: Apr. 28, 2009
(86) PCT No.: PCT/EP2009/003073

§ 371 Date: Nov. 10, 2010

(30) Foreign Application Priority Data
May 10, 2008 (EP) 08008797.6

(51) Int. Cl. A61K 31/5377; A61K 31/506; A61K 31/195; A61P 27/16

(52) U.S. Cl. 514/234.2; 514/256; 514/568

(57) ABSTRACT

The invention provides pharmacological compositions comprising a stimulator or activator of the soluble guanylate cyclase (sGC) either alone or in combination for the treatment of hearing impairment i.e. hearing loss and tinnitus.

### TECHNICAL FIELD OF THE INVENTION

The present invention relates to soluble guanylate cyclase (sGC) and the pharmacology of sGC stimulators and sGC activators. More particularly, the invention relates to the use of sGC stimulators and sGC activators alone and in combination for preparation of medicaments for the treatment of Hearing Impairment, i.e. Hearing Loss and Tinnitus.

### BACKGROUND OF THE INVENTION

Hearing impairment, i.e. Hearing loss and Tinnitus are affecting more than 250 million patients worldwide and are therefore a very common disease. Hearing impairment decrease the quality of life of patients dramatically and could currently not be treated adequately. Hearing loss is often categorized in conductive hearing loss, sensorineural hearing loss, and mixed hearing loss, which is a combination of conductive and sensorineural hearing loss. Conductive hearing loss results from impairment of the external or middle ear, i.e. caused by ear infections. Sensorineural hearing loss includes sensory hearing loss, caused by a cochlea disorder. Neural hearing loss, results from damage of the vestibulocochlear nerve. Most of the cases of hearing loss are sensorineural and caused by i.e. a damage or loss of hair cells in the cochlea. Tinnitus, defined as the perception of sound in the absence of an acoustic stimulus, is often associated with sensorineural hearing loss. The pathophysiology of tinnitus is not well understood. The causes of tinnitus could be similar to the causes of hearing loss, e.g., acoustic trauma, ototoxic drugs, and infections but also includes psychosocial and stress related factors. As noted above, tinnitus is also a symptom of Meniere's disease. Like sensorineural hearing loss, tinnitus is most commonly associated with the inner ear and it is very difficult to treat.

Currently, there are no clinically proven medications for the treatment of tinnitus and hearing loss (sensorineural and neural) and a medication would be very desirable.

Soluble guanylate cyclase (sGC) is a key signal-transduction enzyme that is activated by nitric oxide (NO). Impaired bioavailability and/or responsiveness to endogenous NO have been implicated in the pathogenesis of cardiovascular, endothelial, renal, hepatic, sexual and urological dysfunctions. Correspondingly, nitrates and various ‘NO-donor’ drugs have been used for treating some of these conditions. However, these therapies have important limitations including non-specific interactions of NO with other biomolecules. Compounds that activate sGC in a NO-independent manner might therefore offer a considerable advantage for the therapy of hearing impairment. Two classes of compounds have been identified recently that activate the sGC NO-independently, the heme-dependent sGC stimulators, such as BAY 41-2272, BAY 41-8543, BAY 63-2521, BAY 60-4552 and heme-independent sGC activators, such as BAY 58-2667 and HMR-1766 (for review see Evgenov et al., 2006).

### DISCLOSURE OF THE INVENTION

The term “hearing impairment” refers to a defect in the ability to perceive sound and includes partial hearing loss, complete hearing loss, deafness (complete or partial), The term tinnitus, refers to the perception of non-existent sounds. The hearing impairment may be due to hair cell or neuron damage, wherein the damage is caused by a genetic disorder, loud sounds, ototoxicity, or any other such stressor described in the application. Hearing impairment includes sensorineural hearing loss, conductive hearing loss, combination hearing loss, mild (between 25 and 40 dB), moderate (between 41 and 55 dB), moderately severe (between 56 and 70 dB), severe (between 71 and 90 dB), and profound (90 dB or greater) hearing loss, congenital hearing loss, pre-lingual and post-lingual hearing loss, unilateral (affecting one ear) and bilateral (affecting both ears) hearing loss, or any combination of these, i.e., sensorineural/severe/postlingual/bilateral.

The invention provides sGC stimulators and sGC activators alone or in combination which are useful for the treatment of hearing impairment.

Guanylate cyclase (sGC) stimulator and sGC activator is preferably a compound selected from the group consisting of
2-[1-(2-fluorobenzyl)-1H-pyrazolo[3,4-b]pyridin-3-yl]-5-(4-morpholinyl)-4,6-pyrimidine-diamine (1), described also as example 16 in WO 00/06569, herein incorporated by reference,
2-[1-(2-fluorobenzyl)-1H-pyrazolo[3,4-b]pyridin-3-yl]-5-(4-pyridinyl)-4-pyrimidinamine (2), described also as example 1 in WO 02/42301, herein incorporated by reference,
methyl-4,6-diamino-2-[1-(2-fluorobenzyl)-1H-pyrazolo[3,4-b]pyridin-3-yl]-5-pyrimidinyl-(methyl)carbamate (3), described also as example 8 in WO 03/095451, herein incorporated by reference,
methyl-4,6-diamino-2-[1-(2-fluorobenzyl)-1H-pyrazolo[3,4-b]pyridin-3-yl]-5-pyrimidinyl-carbamate (4), described also as example 5 in WO 03/095451, herein incorporated by reference.
   and
4-({(4-carboxybutyl)[2-(2-{[4-(2-phenylethyl)benzyl]oxy}phenyl)ethyl]amino}methyl)benzoic acid (5)

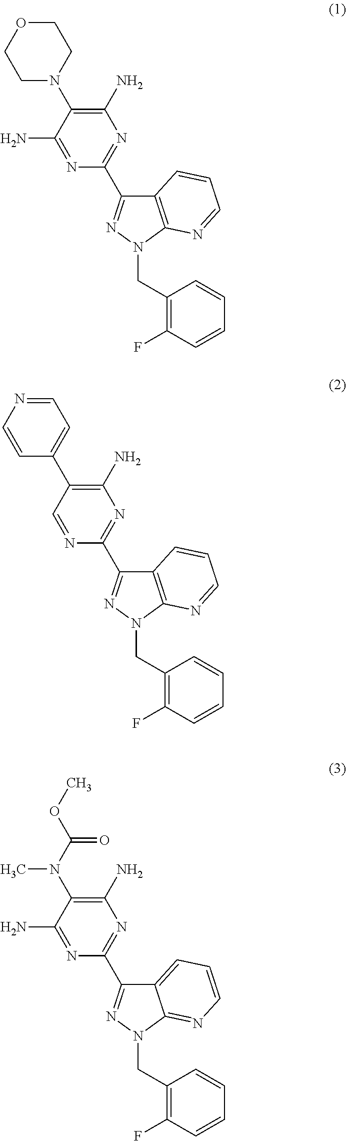

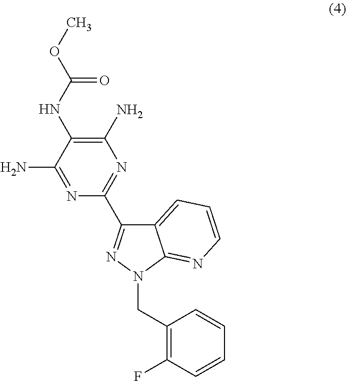

Compounds (1), (2), (3) and (4) are known soluble guanylate cyclase (sGC) stimulators which have been previously described for the treatment of stable angina pectoris or erectile dysfunction.

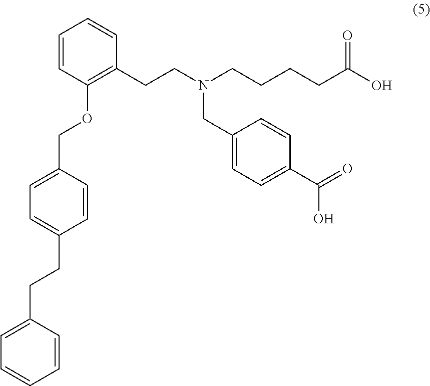

### Compound (5) is Known as sGC Activator

A pharmaceutical composition of the invention is formulated to be compatible with its intended route of administration. Examples of routes of administration include parenteral e.g., intravenous, intradermal, subcutaneous, oral (e.g., inhalation), transdermal (topical) transmucosal and rectal administration. Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents for example, parabens, chlorobutanol, phenol, ascorbic acid, thimerosal, and the like. In many cases, it will be preferable to include isotonic agents, for example, sugars, polyalcohols such as maitol sorbitol sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or con1 starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Bio degradable, biocompatible polymers can be used, such as ethylene vinyl acetate, polyanhydrides, polyglycolic acid, collagen, polyorthoesters, and polylactic acid.

In another embodiment the invention provides sGC stimulators and sGC activators alone or in combination and their use for the preparation of pharmaceutical compositions for the treatment of hearing impairment, whereby these combinations comprise either i) pharmaceutical compositions comprising a compound having a sGC stimulatory action ii) a pharmaceutical compositions comprising a compound having a sGC activatory or iii) pharmaceutical compositions comprising one sGC stimulator and one sGC activator as a fixed combination in one application unit, or iv) a kit of parts containing at least two sets of pharmaceutical compositions, each set consisting of at least one pharmaceutical preparation comprising at least one dose and at least one pharmaceutical preparation comprising a sGC activator or comprising a sGC stimulator in units of at least one dose, whereby each application unit of said pharmaceutical compositions is administered in combination, sequentially, as single dose or in multiple doses.

### In Particular, the Present Invention Provides:

A pharmaceutical composition for the treatment of a disease comprised in a group of diseases consisting of hearing impairment, referring to a defect in the ability to perceive sound including partial hearing loss, complete hearing loss, deafness (complete or partial), and tinnitus.

Hearing impairment, referring to a defect in the ability to perceive sound including partial hearing loss, complete hearing loss, deafness (complete or partial), and tinnitus, in a mammal, comprising a therapeutic agent which regulates the activity of the soluble guanylate cyclase.

A pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal comprising a therapeutic agent which is a stimulator or which is a activator of the soluble guanylate cyclase from the group of sGC activators and stimulators consisting of
2-[1-(2-fluorobenzyl)-1H-pyrazolo[3,4-b]pyridin-3-yl]-5-(4-morpholinyl)-4,6-pyrimidinediamine (1),
2-[1-(2-fluorobenzyl)-1H-pyrazolo[3,4-b]pyridin-3-yl]-5-(4-pyridinyl)-4-pyrimidinamine (2),
methyl-4,6-diamino-2-[1-(2-fluorobenzyl)-1H-pyrazolo[3,4-b]pyridin-3-yl]-5-pyrimidinyl-(methyl)carbamate (3),
methyl-4,6-diamino-2-[1-(2-fluorobenzyl)-1H-pyrazolo[3,4-b]pyridin-3-yl]-5-pyrimidinyl-carbamate (4).
   and
4-({(4-carboxybutyl)[2-(2-{[4-(2-phenylethyl)benzyl]oxy}phenyl)ethyl]amino}methyl)benzoic acid (5)

Use of a sGC stimulator and activator for the preparation of a pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal.

Use of a combination of at least one sGC stimulator and one sGC activator for the preparation of a pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal.

Use of sGC stimulator or activator selected from the group of sGC stimulators and activators of 2-[1-(2-fluorobenzyl)-1H-pyrazolo[3,4-b]pyridin-3-yl]-5-(4-morpholinyl)-4,6-pyrimidinediamine (1), 2-[1-(2-fluorobenzyl)-1H-pyrazolo[3,4-b]pyridin-3-yl]-5-(4-pyridinyl)-4-pyrimidinamine (2), methyl-4,6-diamino-2-[1-(2-fluorobenzyl)-1H-pyrazolo[3,4-b]pyridin-3-yl]-5-pyrimidinyl-(methyl)carbamate (3), methyl-4,6-diamino-2-[1-(2-fluorobenzyl)-1H-pyrazolo[3,4-b]pyridin-3-yl]-5-pyrimidinylcarbamate (4), and 4-({(4-carboxybutyl)[2-(2-{[4-(2-phenylethyl)benzyl]oxy}phenyl)ethyl]amino}methyl)benzoic acid (5) for the preparation of a pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal.

A method for the preparation of a pharmaceutical composition for the treatment of the diseases as mentioned above wherein stimulator and activator of the soluble guanylate-cyclase is selected from the group of compounds consisting of 2-[1-(2-fluorobenzyl)-1H-pyrazolo[3,4-b]pyridin-3-yl]-5-(4-morpholinyl)-4,6-pyrimidinediamine (1),
2-[1-(2-fluorobenzyl)-1H-pyrazolo[3,4-b]pyridin-3-yl]-5-(4-pyridinyl)-4-pyrimidinamine (2), methyl-4,6-diamino-2-[1-(2-fluorobenzyl)-1H-pyrazolo[3,4-b]pyridin-3-yl]-5-pyrimidinyl-(methyl)carbamate (3), methyl-4,6-diamino-2-[1-(2-fluorobenzyl)-1H-pyrazolo[3,4-b]pyridin-3-yl]-5-pyrimidinylcarbamate (4),
   and 4-({(4-carboxybutyl)[2-(2-{[4-(2-phenylethyl)benzyl]oxy}phenyl)ethyl]amino}methyl)benzoic acid (5).

Use of a pharmaceutical composition as mentioned above for the stimulation and activation of the soluble guanylate cyclase in a mammal having a disease comprised in a group of diseases consisting of hearing impairment referring to a defect in the ability to perceive sound including partial hearing loss, complete hearing loss, deafness (complete or partial), and tinnitus in a mammal.

A kit of parts for the treatment of a disease comprised in a group of diseases consisting of hearing impairment referring to a defect in the ability to perceive sound including partial hearing loss, complete hearing loss, deafness (complete or partial), and tinnitus in a mammal, containing a combination of at least one pharmaceutical composition selected from the group of pharmaceutical compositions elected from the group of sGC stimulators and at least one pharmaceutical composition selected from the group of sGC activators.

### In Particular, the Present Invention Provides:

A pharmaceutical composition containing methyl-4,6-diamino-2-[1-(2-fluorobenzyl)-1H-pyrazolo[3,4-b]pyridin-3-yl]-5-pyrimidinylcarbamate (4) and/or 4-({(4-carboxybutyl)[2-(2-{[4-(2-phenylethyl)benzyl]oxy}phenyl)ethyl]amino}methyl)benzoic acid (5), for the treatment of a disease comprised in a group of diseases consisting of hearing impairment.

In order to clarify the effect of sGC stimulators and sGC activators alone and in combination experiments are performed. In particular sGC is semiquantitated, the functional activity of sGC stimulators, sGC activators in vitro is assessed. The functional activity of sGC stimulators and activators in vivo is assessed in model of acoustic trauma (AT) induced hearing loss in rats. The effects of sGC activators and stimulators are semi-quantified on the development, progression and remission of AT-induced hearing impairment. All animal experiments were performed due to the “German Law for the Protection of Laboratory animals” and were conducted due to the approved guidelines for Animal Health and Welfare. Experiments were performed with female Sprague Dawley Rats with a body weight between 300-400g. For induction of acoustic trauma (AT) animals were kept under anesthesia (Ketamine, Xylazin, Rompun i.p. injection) and exposed to band noise or pure tones using a calibrated loudspeaker inside a reverberating chamber. The sound consists of a continuos 10 kHz pure-tone presented at 115 dB SPL. All acoustic stimuli were calibrated at the head level of the animal. Rats were treated with either examples within this invention p.o. dissolved in Ethanol/Solutol/Water (10/40/50) with an application volume of 5 ml/kg or Placebo p.o. [Ethanol/Solutol/Water (10/40/50) with an application volume of 5 ml/kg] twice daily. The first treatment was i.e. 1 h prior to AT. The development and progression/remission of hearing impairment was detected by measuring the hearing thresholds by recordings of auditory brainstem responses (ABR). The threshold was determined by the lowest sound pressure that produced ARBs distinct from noise level. Threshold level analysis was performed prior to the acoustic trauma (AT), 3-5 hours post AT, and on several days post AT (i.e. day 6 post AT).

### REFERENCES

Evgenov O V, Pacher P, Schmidt P M, Haskó G, Schmidt H H H W, Stasch J P. NO-independent stimulators and activators of soluble guanylate cyclase: discovery and therapeutic potential. Nature Rev—Drug Disc. 2006; 5: 755-768.

1-6. (canceled)

7. A method for the treatment of hearing impairment in a mammal comprising the step of administering to a mammal in need thereof a therapeutic agent that regulates the activity of soluble guanylate cyclase (sGC).

8. The method of claim 7, wherein the hearing impairment is a defect in the ability to perceive sound.

9. The method of claim 7, wherein the hearing impairment is partial hearing loss, complete hearing loss, deafness (complete or partial), or tinnitus.

10. The method of claim 7, wherein the therapeutic agent is an sGC stimulator or an sGC activator.

11. The method of claim 7, wherein the therapeutic agent is
2-[1-(2-fluorobenzyl)-1H-pyrazolo[3,4-b]pyridin-3-yl]-5-(4-morpholinyl)-4,6-pyrimidine-diamine (1);
2-[1-(2-fluorobenzyl)-1H-pyrazolo[3,4-b]pyridin-3-yl]-5-(4-pyridinyl)-4-pyrimidinamine (2);
methyl-4,6-diamino-2-[1-(2-fluorobenzyl)-1H-pyrazolo[3,4-b]pyridin-3-yl]-5-pyrimidinyl-(methyl)carbamate (3);
methyl-4,6-diamino-2-[1-(2-fluorobenzyl)-1H-pyrazolo[3,4-b]pyridin-3-yl]-5-pyrimidinyl-carbamate (4); or
4-({(4-carboxybutyl)[2-(2-{[4-(2-phenylethyl)benzyl]oxy}phenyl)ethyl]amino}methyl)benzoic acid (5).

12. The method of claim 7, wherein the therapeutic agent is
methyl-4,6-diamino-2-[1-(2-fluorobenzyl)-1H-pyrazolo[3,4-b]pyridin-3-yl]-5-pyrimidinyl-carbamate (4); or
4-({(4-carboxybutyl)[2-(2-{[4-(2-phenylethyl)benzyl]oxy}phenyl)ethyl]amino}methyl)benzoic acid (5).
